(19) United States
(12) Patent Application Publication
(10) Pub. No.: US 2006/0018949 A1
(43) Pub. Date: Jan. 26, 2006

(54) Injectable biodegradable drug delivery system

(75) Inventors: Daniel M. Ammon JR., Penfield, NY (US); Joseph C. Salamone, Fairport, NY (US); Hiroki Ueda, Houston, TX (US); Seongbong Jo, Houston, TX (US); Antonios G. Mikos, Houston, TX (US)

Correspondence Address: Bausch & Lomb Incorporated, One Bausch & Lomb Place, Rochester, NY, 14604-2701, US

(73) Assignee: Bausch & Lomb Incorporated, Rochester, NY (US)

(21) Appl. No.: 11/079,872

(22) Filed: Mar. 14, 2005

Related U.S. Application Data
(60) Provisional application No. 60/560,059, filed on Apr. 7, 2004.

(51) Int. Cl. A61F 2/00

(52) U.S. Cl. 424/427

(57) ABSTRACT

Compositions for delivering a pharmaceutically active agent to the eye, and/or for treating an ophthalmic disorder, are based on a fumarate polymer, especially a poly(propylene fumarate) polymer.

This invention claims the benefit under 35 USC 119(e) of prior application Ser. No. 60/560,059, filed Apr. 7, 2004.

### FIELD OF THE INVENTION

This invention relates to compositions for delivering a pharmaceutically active agent to the eye, and/or for treating an ophthalmic disorder. The compositions are based on a fumarate polymer, especially a poly(propylene fumarate) polymer.

### BACKGROUND OF THE INVENTION

Various drugs have been developed to assist in the treatment of a wide variety of ailments and diseases. In many instances, such drugs cannot be effectively administered orally without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); and U.S. patent application Ser. No. 10/403,421 (Mosack et al.). Many of these devices include an inner drug core including a pharmaceutically active agent, and some type of holder for the drug core made of an impermeable material such as silicone or other hydrophobic materials. The impermeable holder includes one or more openings for passage of the pharmaceutically agent therethrough to eye tissue.

One polymer that has been successfully used in the general field of medicine is poly(propylene fumarate) (PPF). U.S. Pat. No. 4,888,413 (Domb) describes some of the medical uses for PPF and various methods of synthesizing it. Also, PPF has been described in Biopolymeric Controlled Release Systems Volume II, Donald L. Wise, et al., Chapter 11, 170-184, and in “The Formation of Propylene Fumarate Oligomers for Use in Bioerodible Bone Cement Composites,” by A. J. Domb, et al., Journal of Polymer Science: Part A: Polymer Chemistry, Vol 28, 973-985 (1990). Other disclosures of PPF for medical applications, or methods of synthesizing various polymers of PPF are disclosed in the following, the disclosures of which are incorporated herein by reference: U.S. Pat. No. 5,527,864 (Suggs et al.); U.S. Pat. No. 5,733,951 (Yaszemski et al.); U.S. Pat. No. 6,306,821 (Mikos et al.); U.S. Pat. No. 6,355,755 (Peter et al.); U.S. Pat. No. 6,384,105 (He et al.); U.S. Pat. No. 6,423,790 (He et al.); U.S. patent Publication No. 2002/0,028,189 (Jo et al.); U.S. patent Publication No. 2003/0,032,733 (Fisher et al.); and U.S. patent Publication No. 2003/0,152,548 (Mikos et al.).

### SUMMARY OF THE INVENTION

This invention provides compositions for delivering a pharmaceutically active agent to the eye, and/or for treating an ophthalmic disorder. Additionally, the invention relates to methods employing such compositions.

The drug delivery compositions comprise a matrix of a fumarate polymer and a pharmaceutically active agent, especially a poly(propylene fumarate) polymer and a pharmaceutically active agent. Preferably, the active agent is released from the matrix in a sustained manner over an extended period.

According to a first embodiment, the matrix has the form of a prefabricated or in situ fabricated solid poly(propylene fumarate) polymer loaded with the active agent. The poly(propylene fumarate) may be crosslinked, and this solid matrix may be implanted in an eye of the patient. Alternatively, the poly(propylene fumarate) and the active agent may be co-solved with a crosslinking agent, and this solution may be injected and then crosslinked in an eye of the patient.

According to a second embodiment, the composition has the form of a solution, wherein the active agent is co-solved with a poly(propylene fumarate) polymer in a biocompatible, amphiphilic, and organic solvent, such as N-methylpyrrolidone. The PPF matrix loaded with the active agent can be formed in situ upon injection of the polymer solution into the aqueous environment in an eye of the patient, by dissipation of the amphiphilic organic solvent and precipitation of the polymer which entraps the agent.

According to a third embodiment, the composition includes microspheres or nanospheres comprising the poly(propylene fumarate) polymer loaded with the active agent.

According to another embodiment, the composition comprises a copolymer of poly(propylene fumarate), such as a copolymer of poly(propylene fumarate) and ethylene glycol. The composition may comprise a mixture of poly(propylene fumarate) polymer microspheres loaded with the active agent, and a copolymer of poly(propylene fumarate) loaded with the active agent.

The various embodiments may be injected in the eye of the patient. Alternately, the various compositions may be contained in a holder of a drug delivery device, where the device is implanted in eye tissue.

### BRIEF DESCRIPTION OF THE DRAWING FIGURES

FIG. 1 illustrates cumulative fractional drug release kinetics (expressed as % FA released as a function of time) from non-porous PPF matrices of different formulations of Table 1. 1; n=3±SD.

FIG. 2 illustrates cumulative drug release kinetics (expressed as μg FA released as a function of time) from non-porous PPF matrices of 3.8 mg initial total weight of different formulations of Table 1. 1; n=3±SD.

FIG. 3 illustrates drug release rates (expressed as μg FA released per day as a function of time) from non-porous PPF matrices of 3.8 mg initial total weight of different formulations of Table 1. 1; n=3±SD.

FIG. 4 illustrates cumulative fractional NVP release kinetics (expressed as % NVP released as a function of time) from non-porous PPF matrices of different formulations of Table 1. 1; n=3±SD.

FIG. 5 illustrates cumulative drug release kinetics (expressed as μg FA released as a function of time) from precipitated PPF matrices of 100 mg initial total weight (solvent included) of different formulations of Table 2. 1; n=3±SD.

FIG. 6 illustrates cumulative fractional drug release kinetics (expressed as % FA released as a function of time) from precipitated PPF matrices of different formulations of Table 2. 1; n=3±SD.

FIG. 7 illustrates drug release rates (expressed as μg FA released per day as a function of time) from precipitated PPF matrices of 100 mg initial total, weight (solvent included) of different formulations of Table 2. 1; n=3±SD.

FIG. 8 illustrates cumulative drug release kinetics (expressed as μg FA released as a function of time) from precipitated PPF matrices of 100 mg initial total weight (solvent included) of different formulations of Table 3. 2; n=3±SD.

FIG. 9 illustrates cumulative fractional drug release kinetics (expressed as % FA released as a function of time) from precipitated PPF matrices of different formulations of Table 3. 2;n=3±SD.

FIG. 10 illustrates drug release rates (expressed as μg FA released per day as a function of time) from precipitated PPF matrices of 100 mg initial total weight (solvent included) of different formulations of Table 3. 2; n=3±SD.

### DETAILED DESCRIPTION OF VARIOUS PREFERRED EMBODIMENTS

The drug delivery compositions comprise a matrix of a fumarate polymer, especially a poly(propylene fumarate) polymer, and a pharmaceutically active agent. Various methods of making poly(propylene fumarate) (PPF) polymers are known in the art, including the literature discussed above. Additional methods are illustrated in the examples, below.

According to a first preferred embodiment, the matrix has the form of a prefabricated solid poly(propylene fumarate) polymer loaded with the active agent. These matrices may be prepared by crosslinking PPF in the presence of the active agent.

More specifically, a mixture is first provided, the mixture including PPF and the active agent. Generally, this initial mixture will further include a comonomer and/or a solvent. Since PPF is a hydrophobic polymer, a hydrophobic or amphiphilic carrier (co-monomer or solvent) is generally required to dissolve this polymer.

According to preferred embodiments, this initial mixture includes an amphiphilic monomer. Representative amphiphilic monomers include: (meth)acrylic substituted alcohols, such as 2-hydroxyethyl methacrylate, 2-hydroxyethyl acrylate and glycerol methacrylate; vinyl lactams, such as N-vinylpyrrolidone; and (meth)acrylamides, such as methacrylamide and N,N-dimethylacrylamide.

Optionally, this initial mixture may include a hydrophobic comonomer, either in place of, or in addition to, the amphiphilic co-monomer. Representative hydrophobic co-monomers include: a siloxy-containing monomer, such as a siloxy-containing (meth)acrylate; or an alkyl(meth)acrylate.

Optionally, this initial mixture may include a hydrophilic, non-amphiphilic co-monomer. Representative examples include unsaturated carboxylic acids, such as methacrylic acid and acrylic acid.

As an example, a hydrophobic comonomer will tend to render the resultant solid polymer less permeable to the active agent, whereas a hydrophilic comonomer more permeable to the active agent. Thus, hydrophobic and hydrophilic comonomers may be included, at appropriate ratios, to adjust permeability.

When copolymerizing PPF and the comonomer, the PPF will function as a crosslinking agent, a crosslinking agent being defined as a polymerizable material having multiple polymerizable functionalities. Optionally, a separate crosslinking monomer may be included in the initial monomeric mixture. Examples of crosslinking agents include polyvinyl, typically di- or tri-vinyl monomers, such as di- or tri(meth)acrylates of diethyleneglycol, triethyleneglycol, butyleneglycol and hexane-1,6-diol; divinylbenzene; allylmethacrylate; and bis(4-vinyloxybutyl)adipate.

Preferably, this initial mixture includes a photopolymerization initiator. Typical polymerization initiators include free-radical-generating polymerization initiators of the type illustrated by acetyl peroxide, lauroyl peroxide, decanoyl peroxide, caprylyl peroxide, benzoyl peroxide, tertiary butyl peroxypivalate, sodium percarbonate, tertiary butyl peroctoate, azobis-isobutyronitrile (AIBN); phosphine oxides such as bis(2,4,6-trimethylbenzoyl)phenylphosphine oxide; and acetophenones, such as diethoxyacetophenone.

This initial mixture is added to a mold providing the final shape and configuration of the solid matrix. While contained in the mold, the mixture is polymerized by exposure to light energy, such as a UV light source, or a source of visible light in the blue spectrum. Finally, the resultant solid matrix is removed from the mold.

Preferably, the active agent is included in the matrix in an amount of 0.1 to 10% (w/w), more preferably, 1 to 5% (w/w), based on total weight of the matrix.

This solid matrix may be implanted in an eye of the patient, whereby the active agent is released in a sustained manner over an extended period. A preferred extended period of release is at least one month, and more preferably, at least three months. Especially, this matrix may be implanted in the back of the eye to treat back of the eye disorders such as uveitis, diabetic retinopathy, and diabetic macular edema.

It will be appreciated the dimensions of the solid matrix can vary depending on the specific configuration. The physical size of the device should be selected so that it does not interfere with physiological functions at the implantation site of the mammalian organism. The targeted disease state, type of mammalian organism, location of administration, and agents or agent administered are among the factors which would effect the desired size of the sustained release drug delivery device. However, because the device is intended for placement in the eye, the device is relatively small in size. Generally, it is preferred that the device, excluding any suture tab, has a maximum height, width and length each no greater than 15 mm, more preferably no greater than 10 mm, and most preferably no greater than 3 mm.

Alternately, instead of implanting directly the solid matrix, the solid matrix may be contained in the holder of a drug delivery device, included the devices disclosed in the aforementioned patent literature: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No 6,217,895 (Guo et al.); U.S. Pat. No. 6,375,972 (Guo et al.); and U.S. patent application Ser. No. 10/403,421 (Mosack et al.).

According to another preferred embodiment, the composition has the form of a solution, wherein the active agent is co-solved with PPF polymer in an amphiphilic carrier. These PPF matrices are formed in situ. More specifically, PPF is hydrophobic. By mixing PPF, along with the active agent and an amphiphilic solvent, the PPF will precipitate when dissolved in an aqueous solution, resulting in a matrix of PPF loaded with the active agent. A suitable amphiphilic solvent is N-methylpyrrolidone (NMP) or DMSO. A suitable aqueous solution is saline solution, including saline buffered with a phosphate buffer or a borate buffer.

Optionally, a photoinitiator, such as bis(2,4,6-trimethylbenzoyl)phenylphosphine oxide may be included in the PPF-containing mixture, and then the mixture is crosslinked by exposing the aqueous solution to light energy.

Preferably, the active agent is included in the matrix in an amount of 0.1 to 10% (w/w), more preferably, 1 to 5% (w/w), based on total weight of the matrix.

These solutions, containing PPF loaded with the active agent, may be administered by injection, preferably to eye tissue or tissue surrounding the eye, whereby the active agent is released in a sustained manner over an extended period. Alternately, the solution may be contained in the holder of a drug delivery device, included the aforementioned devices, whereby the holder is implanted in the eye.

When the solution is injected to eye or surrounding tissue, the matrix may be crosslinked photochemically by exposure to light energy at a wavelength of light not harmful to ocular tissue. Alternately, by selecting a gellation temperature of the matrix that approximates the physiological temperature of the eye, the solution may be crosslinked thermally upon injection.

According to a third embodiment, the composition includes microspheres or nanospheres comprising the poly(propylene fumarate) polymer loaded with the active agent.

More specifically, nanospheres or microspheres composed of PPF are biodegradable and incorporate the active agent therein. The microspheres may be prepared by mixing PPF, the active agent, and optionally, a co-monomer and/or a crosslinking agent. This mixture may further include a photoinitiator. The mixture is dissolved in a polar solvent, such as ethyl acetate, and then added to an aqueous solution such as buffered saline solution, and then crosslinked by exposure to light energy. The spheres are recovered by centrifuging and washing the resultant composition.

According to another embodiment, the composition comprises a copolymer of PPF, such as a copolymer of PPF and ethylene glycol. Such copolymers are synthesized by esterification of PPF and PEG at a desired molar ratio. For example, a molar ratio of 1:2 PPF:PEG yields a PEG-PPF-PEG triblock copolymer. By adding the active agent to a solution containing the copolymer at the gellation temperature of the copolymer, the copolymer gels and precipitates, forming a matrix with entrapped active agent. Additionally, by selecting a gellation temperature approximating body temperature, such copolymers (or microspheres or nanospheres) will gel upon injection into the body of a patient.

The spheres or copolymer may be injected into the eye of a patient. Additionally, this injection composition may include an admixture of microspheres or nanospheres, and copolymer. As shown in the examples, below, such as admixture may advantageously result in a slower, sustained release of the active agent.

Ophthalmic disorders treatable with the compositions of this invention include diabetic retinopathy, diabetic macular edema, retinal vascular occlusive disease, uveitis, and choroiditis.

Generally, the active agent may include any compound, composition of matter, or mixture thereof that, when administered to a patient in need thereof, produces a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrase inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasitic agents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors, and cytokine protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitrofurazone, and sodium propionate; antiallergenics such as antazoline, methapyriline, chiorpheniramine, pyrilamine and prophenpyridamine; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; miotics and anti-cholinesterase agents such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

The following examples illustrate various preferred embodiments of the invention.

### EXAMPLE 1

### Sustained Release of Fluocinolone Acetonide from Photo-Crosslinked Poly(propylene fumarate) Matrices

This example investigated the use of prefabricated, non-porous poly(propylene fumarate)-based matrices for the sustained release of the anti-inflammatory drug fluocinolone acetonide for ocular applications. Specifically, poly(propylene fumarate) (PPF)-based matrices were loaded with fluocinolone acetonide (FA), where the matrices include N-vinylpyrrolidone (NVP) as an amphiphilic co-monomer and are crosslinked by photopolymerization.

PPF was synthesized by transesterification of diethyl fumarate and propylene glycol according to methods known in the art. (Shung, A. K., et al., J. Biomater. Sci. Polym. Ed. 13, 95-108 (2002), the disclosure of which is incorporated herein by reference.) FA-loaded non-porous PPF matrices were prepared by photo-crosslinking of PPF and NVP in the presence of FA (Timmer, M. D., et al., Biomacromolecules 4, 1026-1033 (2003), the disclosure of which is incorporated herein by reference.) Bis(2,4,6-trimethylbenzoyl)phenylphosphine oxide (BAPO) was added as a photoinitiator. After injection of the mixture into a silicone mold and crosslinking with a dental blue light, the dimensions of the resultant cylindrical PPF matrices were 1 cm long by 0.6 mm in diameter (3.8 mg). By altering the drug loading (5 or 10 wt %) and ratio of NVP/PPF (0.33 or 0.67), four test formulations of PPF matrices were examined, as reported in Table 1.1 below.

TABLE 1.1
Compositions of the examined formulations.

| | Group 1 | Group 2 | Group 3 | Group 4 |
|---|---|---|---|---|
| NVP/PFF | 0.67 | 0.67 | 0.33 | 0.33 |
| FA wt % | 10 | 5 | 10 | 5 |

For release experiments, samples were immersed in phosphate buffered saline (PBS) (2 ml) on a shaker table (75 rpm) at 37° C. The supernatant was collected periodically and replaced with fresh PBS. The amount of released drug and unreacted NVP at specified time intervals were determined by high performance liquid chromatography (HPLC) at 238.1 nm and 233 nm respectively. The mobile phase for HPLC analysis was methanol:water (7:3).

The amount of drug released during the first day of the experiment was reported as the initial burst release. The drug release rate was determined from the average of the slope of the release curve between three consecutive data points, and is expressed as μg FA per day. All experiments were conducted in triplicate and the results are reported as means ± standard deviation (SD). All the data were statistically analyzed using Dunnett's multiple comparison procedure, and statistical significance was accepted at p<0.05.

The number average molecular weight of the PPF was determined to be 1770 by gel permeation chromatography using polystyrene standards. FA could be incorporated into PPF matrices crosslinked with NVP up to 10 wt % of polymer mass.

An initial burst release of less than 6% of the incorporated drug was observed (Table 1. 2). Increasing the NVP content significantly reduced the burst release due to enhanced crosslinking of the PPF matrices (Table 1. 2). In contrast, greater drug loading significantly increased the burst release by enhancing the diffusivity of the drug and reducing the crosslinking density of the PPF matrix (Table 1. 2).

TABLE 1.2
Initial burst release of FA from PPF matrices.

| | Group 1 | Group 2 | Group 3 | Group 4 |
|---|---|---|---|---|
| FA Initial Burst (%) | 2.3 ± 0.5 | 0.6 ± 0.4 | 5.8 ± 0.4 | 2.4 ± 0.8 |

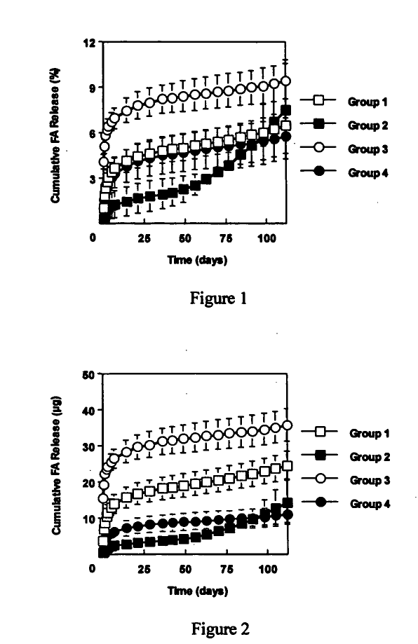

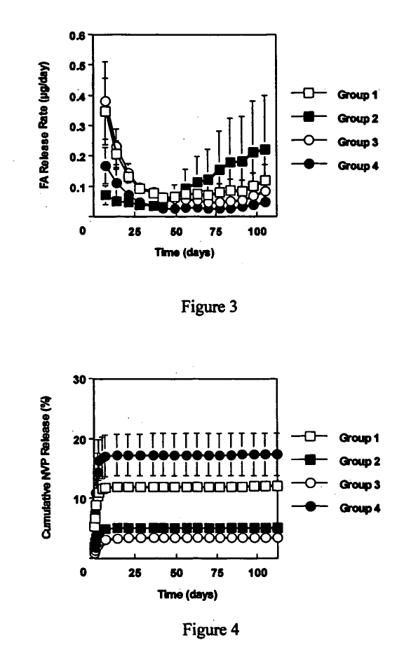

After the initial burst, the drug release was sustained over 16 weeks (FIGS. 1 and 2). During the first 7 weeks, increasing the NVP content resulted in reduced drug release rate, and greater drug loading significantly increased the drug release rate by similar mechanisms as for the burst release (FIG. 3). After 9 weeks of release, the FA release rates for 3 formulations were constant over time, except for Group 2, which exhibited increased FA release rates.

Most of the uncrosslinked NVP was released within a week and was less than 20% of the initial NVP (FIG. 4).

FA could be incorporated into PPF matrices crosslinked with NVP up to 10 wt % of polymer mass and could be released gradually over 16 weeks. The release kinetics could be modified by changing the drug loading and the ratio of NVP/PPF. These results support the feasibility of sustained release of FA from photo-crosslinked PPF-based non-porous matrices for ocular applications where the (PPF)-based matrices loaded with fluocinolone acetonide (FA) are implanted in the eye, especially in the back of the eye, and FA is released to the eye region of implantation.

### EXAMPLE 2

### Controlled Release of Fluocinolone Acetonide from in Situ Forming Poly(propylene fumarate) Matrices

This example investigated the use of the degradable polyester poly(propylene fumarate) (PPF) as part of an injectable carrier for controlled release of the drug fluocinolone acetonide (FA) for ocular applications. In this experiment, in situ forming delivery systems comprised of linear PPF, FA, and N-methylpyrrolidone (NMP) were fabricated, and FA loading dosage to in vitro release kinetics over a period of 15 weeks was determined. The effects of NMP content and surface photo-crosslinking on in vitro release kinetics were also evaluated over a period of 15 weeks.

PPF was synthesized by transesterification of diethyl fumarate and propylene glycol similar to Example 1. FA-loaded PPF matrices were prepared by dissolving PPF and FA in NMP and then injecting the solution into phosphate buffered saline (PBS) using a syringe pump. Four test formulations were prepared by varying the drug content (2.5 and 5.0 wt % FA) while keeping the polymer content constant (Groups 1 and 2 in Table 2. 1), by changing the solvent content (Group 3 in Table 2. 1), or by adding a photo-initiator, bis(2,4,6-trimethylbenzoyl)phenylphosphine oxide (BAPO) (Group 4 in Table 2. 1).

PPF matrices were fabricated by injecting 30 μl of the polymer/drug solution into 10 ml of PBS (at a rate of 1 μl/sec) resulting in precipitation of the hydrophobic PPF, thus forming a matrix including entrapped FA. After injection of the mixture into PBS, the samples of Group 4 were crosslinked by exposing under a dental blue light, similar to Example 1.

TABLE 2.1
Compositions of examined formulations

| | Group 1 | Group 2 | Group 3 | Group 4 |
|---|---|---|---|---|
| PPF wt % | 50.0 | 50.0 | 25.0 | 50.0 |
| FA wt % | 5.0 | 2.5 | 2.5 | 5.0 |
| NMP wt % | 45.0 | 47.5 | 72.5 | 44.5 |
| BAPO wt % | 0 | 0 | 0 | 0.5 |

For drug release studies, PPF matrices were stirred in PBS (10 ml) on a shaker table (75 rpm) at 37° C. The supernatant was collected periodically and replaced with fresh PBS. The amount of released drug at specified time intervals was determined by high performance liquid chromatography (HPLC). The mobile phase for HPLC analysis was methanol:water (7:3), and the wavelength for FA detection was 238.1 nm.

The amount of drug released during the first day of the experiment was reported as the initial burst release. The drug release rate was determined from the average of the slope of the release curve between three consecutive data points, and is expressed as μg FA per day. The theoretical release time corresponding to complete drug release was calculated from the drug release rate during the period of days 7 to the last day by extrapolation. All studies were conducted in triplicate and the results are reported as means ± standard deviation.

The number average molecular weight of the PPF was 2800 as measured by gel permeation chromatography using polystyrene standards. A PPF matrix loaded with FA was formed upon injection of the PPF and FA solution in NMP into PBS and the dissipation of NMP into PBS.

An initial burst release varying from 22 to 68% of the incorporated drug was observed (Table 2. 2). Greater drug loading significantly reduced the burst release due to skin formation at the precipitation of the matrices (Table 2. 2). In contrast, increasing solvent content or decreasing drug content significantly increased the burst release by enhancing the diffusivity of the drug, which was entrapped in residual solution phase (Group 2 in Table 2. 2). The surface photo-crosslinking did not affect the initial burst significantly (Group 4 in Table 2. 2).

TABLE 2.2
Release characteristics of FA from PPF matrices.

| | Group 1 | Group 2 | Group 3 | Group 4 |
|---|---|---|---|---|
| FA initial burst (%) | 22.2 ± 1.5 | 42.4 ± 0.6 | 68.2 ± 3.0 | 23.6 ± 5.2 |
| FA release time (months) | 12.8 ± 7.4 | 4.6 ± 1.1 | 6.1 ± 1.0 | 11.2 ± 3.9 |

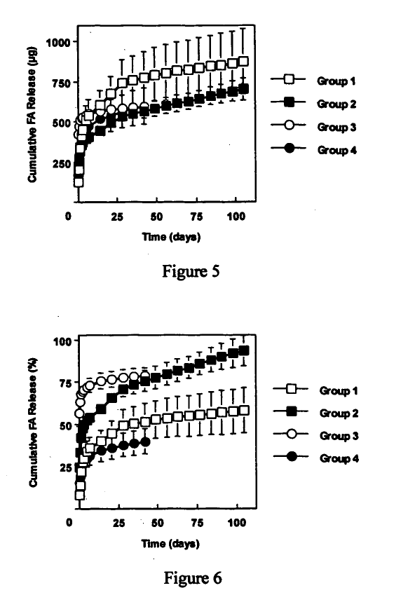

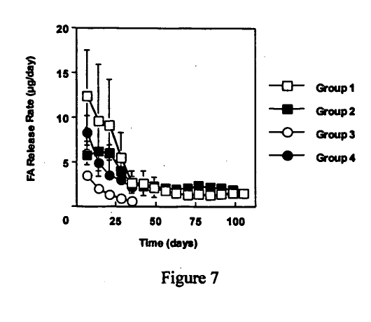

After an initial burst release, the drug was gradually released for more than a month (FIGS. 5 and 6). Increasing the drug content increased the drug release rate (FIG. 7). After 112 days, 59% and 95% of the total drug were released from groups 1 and 2, respectively (FIG. 6). In contrast, increasing solvent content or decreasing drug content decreased the drug release rate by depletion of the drug at the large initial burst release (Group 2 in FIG. 6). The surface photo-crosslinking decreased the drug release rate by suppressing the diffusivity of the drug (Compare Group 4 with Group 1 in FIG. 6).

These results support the potential of PPF-based injectable, in situ forming drug delivery systems for ocular applications, where FA is released from the system after injection of the system in eye tissue upon degradation of PPF.

### EXAMPLE 3

### Controlled Release of Fluocinolone Acetonide from in Situ Forming Poly(propylene fumarate-co-Ethylene Glycol) Matrices Incorporating Poly(propylene fumarate) Microspheres

In this example, microspheres (MS) composed of a biodegradable polyester poly(propylene fumarate) (PPF) incorporating the anti-inflammatory drug, fluocinolone acetonide (FA), are synthesized. Also, a poly(ethylene glycol)-poly(propylene fumarate)-poly(ethylene glycol) (PEG-PPF-PEG) tri-block copolymer (CP) exhibiting thermoreversible properties was synthesized, for use as an injectable, in situ forming hydrogel carrier. The in vitro release kinetics of FA from copolymer, copolymer with MS, and MS in phosphate buffered saline (PBS) over a period of 8 weeks was investigated.

PPF was synthesized by transesterification of diethyl fumarate and propylene glycol, similar to Example 1. FA loaded microspheres were prepared as follows. PPF, FA, bis(4-vinyloxybutyl) adipate (VOBA) as a crosslinking agent, bis(2,4,6-trimethylbenzoyl)phenylphosphine oxide (BAPO) as a photo-polymerization initiator, and a small amount of PPF-PEG copolymer as a surfactant were dissolved in ethyl acetate (Table 3. 1). The polymer solution was poured into phosphate buffered saline (PBS), mixed rigorously to make the suspension, and finally crosslinked by exposing under a dental blue-light, similar to Example 1. The obtained microspheres were centrifuged, and washed repeatedly with water, and dried. In Table 3.1, the reported weight percent of each component of MS is total mass of the polymer without the solvents, which were removed from the obtained carriers in the drying process.

TABLE 3.1
Compositions of FA loaded PPF microspheres.
Components of MS*

| | |
|---|---|
| PPF wt % | 45.5 |
| FA wt % | 5.27 |
| VOBA wt % | 44.9 |
| PPF-PEG copolymer wt % | 3.7 |
| BAPO wt % | 5.9 |

Solvents

| | |
|---|---|
| Ethyl acetate (ml) | 3.0 |
| Water (ml) | 30.0 |

The copolymer was synthesized by esterification of PPF and PEG at the molar ratio of one to two. Copolymer matrices were fabricated by injecting 100 μl of the polymer solution containing FA or FA loaded microspheres into 5 ml of PBS at 37° C. resulting in thermal gelation and precipitation of copolymer, thus forming a matrix including entrapped FA (Table 3. 2).

TABLE 3.2
Compositions of examined formulations.

| | CP | CP + MS | MS |
|---|---|---|---|
| CP wt % | 25.0 | 25.0 | 0 |
| FA wt % | 0.1^{*1} | 0.5^{*1} | 5.3 |
| MS wt % | 0 | 10.0^{*2} | 100^{*2} |
| Glucose wt % | 10.0 | 10.0 | 0 |
| PBS wt % | 64.9 | 55.0 | 0 |

^{*1}The weight percent of FA is reported as total mass of the carrier including aqueous phase incorporated in the CP hydrogels.
^{*2}The weight percent of MS including FA is reported as total mass of the carrier including aqueous phase incorporated in the CP hydrogels.

For drug release studies, the obtained copolymer matrices and microspheres were stirred in PBS (5 ml) on a shaker table (75 rpm) at 37° C. (Table 3. 2). Two ml of the supernatant was collected periodically and replaced with fresh PBS. To dissolve the micelle formed by copolymer in aqueous environment, 15 wt % of ethanol was added to the samples from copolymer systems. The amount of released drug at specified time intervals was determined by high performance liquid chromatography (HPLC). The mobile phase for HPLC analysis was methanol:water (7:3), and the wavelength for FA detection was 238.1 nm.

The amount of drug released during the first day of the experiment was reported as the initial burst release. The drug release rate was determined from the average of the slope of the release curve between three consecutive data points, and is expressed as μg FA per day. The theoretical release time corresponding to complete drug release was calculated from the drug release rate during the period of days 7 to the last day by extrapolation. All studies were conducted in triplicate and the results are reported as means ± standard deviation.

The number average molecular weight of the PPFs was 2800 as measured by gel permeation chromatography using polystyrene standards. This PPF was used to synthesize both the copolymer and microspheres.

The reaction yield of copolymer synthesis calculated from the GPC data was 55%. To obtain the transition temperature of the copolymer less than 37° C., 25 wt % of copolymer and 10 wt % of glucose in the copolymer solution were required. A copolymer matrix loaded with FA or FA incorporated microspheres was formed upon injection of the copolymer mixtures into PBS at 37° C. and thermal gelation of copolymer. FA could be incorporated into copolymer matrix up to 0.1 wt % of total mass of the carriers, which was 18 times concentrated than FA saturated aqueous solution without copolymer.

The diameter of microspheres prepared with PPF, VOBA, and ethyl acetate was measured by scanning electron microscopy to be 30 μm, which was significantly smaller than that of microspheres prepared with N-vinylpyrrolidone as the crosslinking agent and methylene chloride as the solvent (130 μm). By the extraction test of microspheres using tetrahydrofuran, the incorporating efficiency of FA in microspheres was calculated to be 41.0%. FA could be incorporated into the microspheres up to 5.3 wt % of total mass of the polymer.

The drug release mechanisms from all three groups are considered to be based on the hydrolysis of the polymer and the diffusion of the drug to the outer solution. An initial burst release varying from 38 to 76% of the incorporated drug was observed (Table 3. 3). The large burst release from the copolymer could be caused by high swelling ratio of the polymer in water and low affinity to the hydrophobic drug (Table 3. 3). The breakage of the copolymer hydrogel owing to its low physical strength also caused the drug burst release, when the turbulence happened by stirring and exchanging of the supernatant. The large burst release from the microspheres was probably due to release of any FA which was not complexed with the PPF during the microspheres loading. Some precipitated drug on the surface of the microspheres was observed by SEM (data not shown). In contrast, significantly smaller initial burst release of the drug was observed in using the copolymer-microspheres composite than either copolymer or microspheres alone (Table 3. 3). This was considered to be due to the suppression of drug diffusion by the polymer network and the amphiphilicity of the copolymer matrix.

TABLE 3.3
Release characteristics of FA from PPF matrices.

| | CP | CP + MS | MS |
|---|---|---|---|
| FA initial burst (%) | 73.0 ± 2.6 | 38.2 ± 2.0 | 76.1 ± 2.3 |
| FA release time (months) | 2.5 ± 1.6 | 3.6 ± 0.8 | 0.67 ± 0.31 |

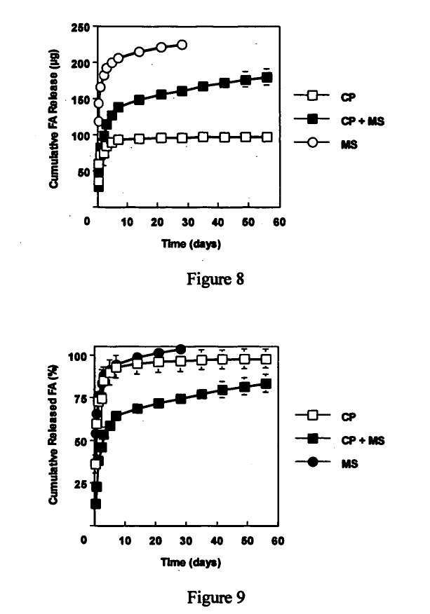

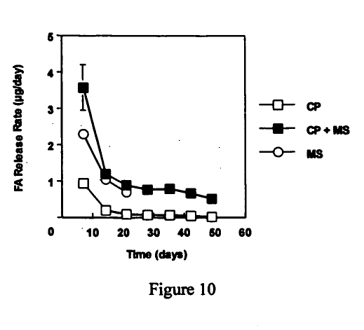

Only by using copolymer+microspheres (CP+MS), the drug was gradually released for more than 2 months after an initial burst release (FIGS. 8 and 9). From the microspheres, the incorporated drug was released 90% in a week, and 100% in a month (FIG. 9). From the copolymer, the drug was released 90% and reached a plateau in a week (FIG. 9). The copolymer and microspheres showed lower drug release rate than CP+MS (FIG. 10). This could be caused by the loosening of the polymer network in CP+MS owing to the incorporation of microspheres and by the exhaustion of the drug owing to the large initial burst in copolymer and microspheres.

These results support the potential of PPF microspheres and injectable, in situ physical forming PPF-based copolymer matrices for the intraocular drug delivery applications.

The examples and illustrated embodiments demonstrate some of the sustained release embodiments of the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What we claim is:

1. A drug delivery composition for placement in the eye, comprising a matrix of fumarate polymer and a pharmaceutically active agent.

2. The drug delivery composition for placement in the eye, comprising a matrix of poly(propylene fumarate) polymer and a pharmaceutically active agent.

3. The composition of claim 2, wherein the poly(propylene fumarate) is crosslinked.

4. The composition of claim 3, wherein the poly(propylene fumarate) is copolymerized with an amphiphilic or a hydrophobic monomer.

5. The composition of claim 2, wherein the matrix has the form of a prefabricated solid poly(propylene fumarate) polymer loaded with the active agent.

6. The composition of claim 5, wherein the prefabricated solid has a maximum height, width and length each no greater than 15 mm.

7. The composition of claim 2, wherein the matrix has the form of a solution, which may be injected in the eye and crosslinked in situ to form a solid poly(propylene fumarate)polymer loaded with the active agent.

8. The composition of claim 7, wherein the matrix is crosslinked photochemically by exposure to light energy at a wavelength of light not harmful to ocular tissue, or thermally at a physiological temperature of the eye.

9. The composition of claim 1, wherein the active agent is released from the matrix in a sustained manner.

10. The composition of claim 2, having the form of a solution, wherein the active agent is co-solved with a poly(propylene fumarate) polymer in an amphiphilic carrier.

11. The composition of claim 1, wherein the composition includes microspheres or nanospheres comprising the fumarate polymer loaded with the active agent.

12. The composition of claim 11, wherein the composition comprises microspheres including a copolymer of poly(propylene fumarate).

13. The composition of claim 12, wherein the composition comprises the microspheres and an aqueous carrier.

14. The composition of claim 2, comprising a copolymer of poly(propylene fumarate).

15. The composition of claim 14, wherein the composition comprises a copolymer of poly(propylene fumarate) and ethylene glycol.

16. The composition of claim 1, wherein the composition comprises a mixture of poly(propylene fumarate) polymer microspheres loaded with the active agent, and a copolymer of poly(propylene fumarate) loaded with the active agent.

17. The composition of claim 16, wherein the composition further comprises an aqueous carrier.

18. The composition of claim 1, having a transition temperature approximating body temperature of a patient.

19. The composition of claim 18, having a transition temperature less than about 37° C.

20. A method of treating ophthalmic disorders, comprising administering to a patient a composition comprising a matrix of a fumarate polymer and a pharmaceutically active agent.

21. The method of claim 20, wherein the composition comprises a matrix of poly(propylene fumarate) polymer and a pharmaceutically active agent.

22. The method of claim 21, wherein the composition has the form of a prefabricated solid matrix of poly(propylene fumarate) polymer loaded with the active agent.

23. The method of claim 22, wherein the solid matrix is implanted in an eye of the patient.

24. The method of claim 22, wherein the solid matrix is implanted at a back portion of the eye.

25. The method of claim 20, wherein the solid matrix is injected in an eye of the patient.

26. The method of claim 20, wherein the poly(propylene fumarate) polymer is crosslinked.

27. The method of claim 20, wherein the active agent is released from the matrix in a sustained manner.

28. The method of claim 20, wherein the composition has the form of a suspension, wherein the active agent is entrapped in a poly(propylene fumarate) polymer, and the polymer is suspended in an aqueous carrier.

29. The method of claim 20, wherein the composition includes microspheres or nanospheres comprising the poly(propylene fumarate) polymer loaded with the active agent.

30. The method of claim 20, wherein the composition includes microspheres comprising the poly(propylene fumarate) polymer loaded with the active agent.

31. The method of claim 30, wherein the microspheres comprise a copolymer of poly(propylene fumarate).

32. The method of claim 30, wherein the composition includes the microspheres and an aqueous carrier.

33. The method of claim 29, wherein the composition comprises a mixture of poly(propylene fumarate) polymer microspheres loaded with the active agent, and a copolymer of poly(propylene fumarate) loaded with the active agent.

34. The method of claim 20, wherein the composition comprises a copolymer of poly(propylene fumarate).

35. The method of claim 34, wherein the composition comprises a copolymer of poly(propylene fumarate) and ethylene glycol.

36. The method of claim 20, wherein the composition is injected in eye tissue of the patient, and has a transition temperature approximating a body temperature of the patient, whereby the composition is crosslinkable in situ upon injection.

37. The method of claim 36, where the composition has a transition temperature less than about 37° C.

38. A method comprising delivering to eye tissue a composition comprising a matrix of fumarate polymer and a pharmaceutically active agent.

39. The method of claim 38, comprising delivering to eye tissue a composition comprising a matrix of poly(propylene fumarate) polymer and a pharmaceutically active agent.

40. The method of claim 38, wherein the composition is implanted in eye tissue.

41. The method of claim 38, wherein the composition is injected in eye tissue.

42. The method of claim 38, wherein the composition is contained in a holder of a drug delivery device, and the device is implanted in eye tissue.

43. The method of claim 38, wherein the composition is contained in a holder of a drug delivery device, and the device is injected in eye tissue.
